(19) United States
(12) Patent Application Publication
(10) Pub. No.: US 2025/0057512 A1
(43) Pub. Date: Feb. 20, 2025

(54) Cartridge for Urine Test

(71) Applicant: Hak Soo Kim, San Diego, CA (US)

(72) Inventor: Hak Soo Kim, San Diego, CA (US)

(21) Appl. No.: 18/800,165

(22) Filed: Aug. 12, 2024

(30) Foreign Application Priority Data
Aug. 14, 2023 (KR) 10-2023-0106300
Sep. 26, 2023 (KR) 10-2023-0129724

(51) Int. Cl. A61B 10/00; A61B 5/145; A61B 50/30

(52) U.S. Cl. CPC A61B 10/007; A61B 5/14507; A61B 50/30; A61B 2010/0006; A61B 2562/0295; A61B 2562/08

(57) ABSTRACT

The present invention relates to a urine test cartridge. The urine test cartridge comprising a urine test strip having at least one sector connected to it that discolors upon contact with urine, a protective cover removably attached thereto, and a reel on which the urine test string is wound in a circular shape.

### CROSS-REFERENCE TO RELATED APPLICATIONS

Any and all priority claims identified in the Application Data Sheet, or any correction thereto, are hereby incorporated by reference under 37 CFR 1.57. This application claims the benefit of South Korean Provisional Application No. 10-2023-0106300 filed on Aug. 14, 2023, South Korean Non-Provisional Application No. 10-2023-0129724 filed on Sep. 26, 2023, and South Korean Issued Patent No. 10-2643175 on Feb. 28, 2024, respectively. The aforementioned applications are incorporated by reference herein in their entirety, and are hereby expressly made a part of this specification.

### FIELD OF THE INVENTION

The present invention relates to a cartridge for urine test, which is inserted into and used in a urine test device capable of automatically testing urine, and which is wound on a reel with urine test strips having sectors connected thereto that may be used multiple times without having to be replaced after each use, and which may easily communicate information to the urine test device through a barcode area on the urine test strips.

### BACKGROUND OF THE INVENTION

Urine is blood filtered through the kidneys, so substances in the blood are found in the urine. Because it is relatively easy to collect urine, urine tests are used as basic reference data to check for diseases and health conditions, pregnancy, urinary tract infections, and psychiatric or metabolic disorders such as kidney or liver problems. Failure to check blood sugar periodically may lead to serious diseases such as chronic kidney disease, so it is important to make urine tests more accessible so that users may test themselves from time to time to check their health status.

Not only do typical personal urine test strips change in their properties over time, resulting in inaccurate measurements, but also they become significantly less accurate if they are not tested within a specified time after urine is applied. In addition, when inspecting with the naked eye, it is difficult to compare accurately with the standard color table because of the differences in color perceived by each individual. Using the camera of a wireless phone to analyze urine with a standard color display increases the accuracy compared to the naked eye, but if the ambient lighting environment, tilt of the phone, distance to the test strip, etc. are not correct at the time of filming, the color of the elements that the urine has come into contact with may change and the accuracy of the standard color display may decrease. In addition, you may visit a hospital/clinic or urine collection place to submit urine and send it to an institution with testing equipment for testing, but this is inconvenient in that it costs a lot of money and time. It would be much more convenient to manage your health at home if you could install a urine test device on the toilet at home and analyze your urine immediately. Additionally, you may conveniently check accurate test results by using urine test strips that may be used continuously instead of using individual strips that need to be replaced after each use.

The prior invention includes Korean Patent Publication No. 20-0470398 (Strip for Urine Test), in which the camera may simultaneously photograph the degree of discoloration of the reagent pad part of the strip that reacts with urine and the standard color display consisting of three primary colors of light displayed on one side of the strip, and compare the colors of the images taken through the app., and based on the degree of discoloration of the changed color, the test results may be displayed by color according to the components contained in the urine, and depending on the degree of discoloration of each color, it is only possible to perform a simple and convenient urine test to detect various diseases including glucose metabolism management, kidney function management, liver function management, and urinary tract infection management early.

### Problem to be Solved

The problem to be solved by the present invention is to provide a cartridge for urine test that may be conveniently used for a long period of time in the form of a cartridge in which several strips are connected and wound together without using a single strip for each urine test, and that may be used for a long period of time without degeneration by being protected from moisture or air due to a protective cover on the top of the urine test strip.

### Means to Solve Technical Problems

The present invention relates to a urine test cartridge, comprising a urine test strip having at least one sector connected to it that discolors upon contact with urine, a protective cover removably attached thereto, and a reel on which the urine test string is wound in a circular shape.

### Effects of the Invention

The present invention utilizes a cartridge with multiple strips connected to it, which enables convenient long-term use without the need to change urine test strips after each urine test.

Also, the top of the urine test strip has a protective cover to protect it from moisture and air, so it may be used for a long time without degeneration.

In addition, the barcode area on the urine test strip may easily communicate information to the urine test device.

### BRIEF DESCRIPTION OF THE DRAWINGS

FIG. 1 is a diagram illustrating an overall view of a urine test cartridge according to an embodiment of the present invention.

FIGS. 2A and 2B are a front and side view of a reel according to an embodiment of the present invention.

FIG. 3 is a top view of a urine test strip according to an embodiment of the present invention.

FIG. 4 is a top view of a sector according to an embodiment of the present invention.

FIG. 5 is a top view of another sector according to another embodiment of the present invention.

FIG. 6 is a cross-sectional view of a urine test strip according to an embodiment of the present invention.

FIG. 7 is a cross-sectional view of a urine test strip according to another embodiment of the present invention.

FIG. 8 is a perspective view of a urine test strip according to an embodiment of the present invention.

### DETAILED DESCRIPTION OF EXEMPLARY EMBODIMENTS

Any specific structural or functional descriptions of embodiments according to the concepts of the present invention disclosed herein are exemplified merely for the purpose of illustrating embodiments according to the concepts of the present invention, and embodiments according to the concepts of the present invention may be implemented in various forms and shall not be limited to the embodiments described herein.

Since the embodiments according to the concepts of the present invention may be subject to various modifications and may take many forms, the embodiments are illustrated in the drawings and described in detail herein. However, this is not intended to limit the embodiments according to the concepts of the present invention to any specific disclosed forms, and shall include all modifications, equivalents, or substitutions that are within the scope of the ideas and techniques of the present invention.

The terminology used in this specification is used to describe specific embodiments only and is not intended to limit the present invention. Singular expressions shall include plural expressions unless the context clearly dictates otherwise. In this specification, the terms “include/comprise” or “have” and the like are intended to indicate the presence of the features, numbers, steps, operations, components, parts, or combinations thereof described herein, and are not to be understood as precluding the possibility of the presence or addition of one or more other features, numbers, steps, operations, components, parts, or combinations thereof.

Hereinafter, embodiments of the present invention will be described in detail with reference to the drawings attached to this specification.

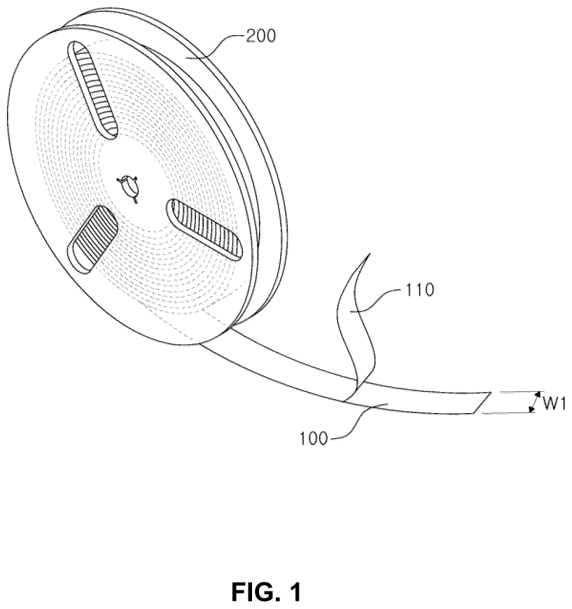

FIG. 1 is a diagram illustrating an overall view of a urine test cartridge according to an embodiment of the present invention.

Referring to FIG. 1, the urine test cartridge (10) comprises a urine test strip (100) with a protective cover (110) removably attached thereto and a reel (200) on which the urine test string (100) is wound in a circular shape.

The protective cover (110) may be spaced apart from the sample area at a certain distance to avoid direct contact with the sample on the urine test strip (100). The protective cover (110) may be formed to be attached to the urine test strip (100) and removable from within the urine test device. Additionally, with the protective cover (110) attached and not removed from the inside of the urine test device, a needle may be used to penetrate the protective cover (110) in the sample area and inject urine. In addition, in the case of a urine test cartridge used in a urine test device installed in a toilet, the protective cover (110) is made of a material that dissolves quickly in water and does not modify the properties of the sample on the urine test strip (100), so that the protective cover (110) dissolves immediately after contact with the urine, and the urine test may proceed without removing it separately. The protective cover (110) is to prevent the urine test strip (100) from being oxidized by moisture or air, and in the case of a urine test cartridge used in a urine test device installed in a toilet, the protective cover (110) is formed of a material that is soluble in water so that it may be disposed of when flushing the toilet. The protective cover (110) may be formed of a transparent material to allow the urine test device to accurately recognize at least one of the barcode area and the standard color display of the urine test strip (100), but is not necessarily limited thereto.

The urine test strip (100) may have at least one sector connected to the test strip (100) to test for color change over time as urine comes into contact with the sample area. The urine test strip (100) may be formed of at least one of, but not necessarily limited to, paper or tape that is soluble in water for environmental-friendliness. The urine test strip (100) may include a barcode area that provides information on the urine test strip (100), which will be described later. W1 denotes a width of the urine test strip (100). The smaller the width (W1) of the urine test strip (100), the smaller the volume of the urine test device.

The reel (200) may be wrapped around the urine test strip (100) in a circular shape, but need not necessarily be circular and may be wrapped around the urine test strip (100) in a wrinkle-free manner so as not to interfere with the barcode area and the sample area of the urine test strip (100). The reel (200) may be wrapped around the urine test strip (100) such that the sample area of the urine test strip (100) is located on the inside, but is not necessarily limited thereto. A detailed structure of the reel (200) will be described later.

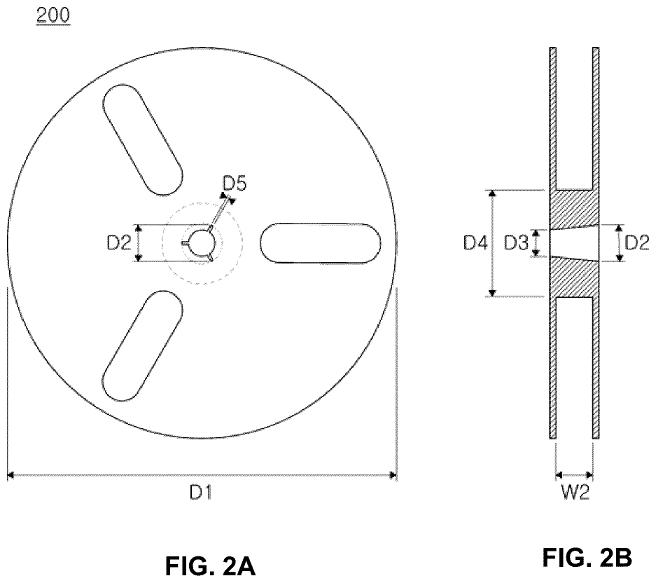

FIG. 2 is a front and side view of a reel according to an embodiment of the present invention.

Referring to FIG. 2A, D1 represents an outer size of the reel (200). The outer size (D1) of the reel (200) is preferably small for miniaturization of the urine test device and the urine test cartridge (10), but a smaller D1 may reduce the amount of urine test strip (100) that is wound onto the reel (200), which may result in fewer urine tests being available at one time. Referring to FIG. 2B, D2 represents an inner size of the fixing hole of the reel (200), and D3 represents an outer size of the fixing hole of the reel (200). The inner size (D2) of the fixing hole of the reel (200) is designed to be larger than the outer size (D3) of the fixing hole of the reel (200), so that the reel (200) may be easily mounted on the urine test device. D4 represents the inner side of the reel 200. The inner size (D4) of the reel (200) is preferably small to accommodate a large amount of urine test strips (100), but a smaller D4 may cause the urine test strips (100) to be wrinkled or damaged. D5 represents a hole through which the reel (200) is mounted to the urine test device. The reel (200) on which the urine test strip (100) is wound is mounted to the urine test device so that the reel (200) rotates to unwind the urine test strip (100) during the urine test. W2 refers to the inner width of the reel 200. The inner width (W2) of the reel (200) may be designed to be slightly larger than the width (W1) of the urine test strip (100).

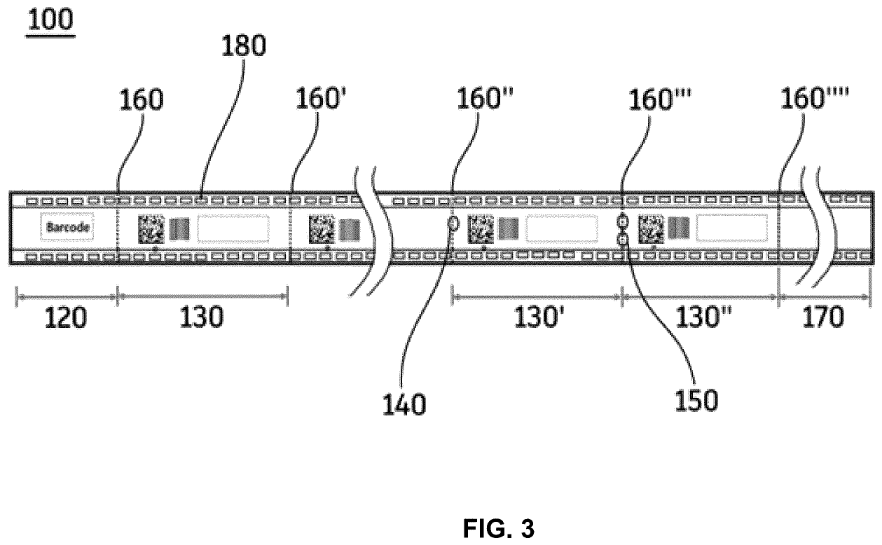

FIG. 3 is a top view of a urine test strip according to an embodiment of the present invention.

Referring to FIG. 3, the urine test strip (100) comprises a first barcode area (120), sector (130), a first mark (140), a second mark (150), separation line (160), spare area (170), and hole (180).

The first barcode area (120) may include a barcode comprising information about the urine test strip (100), and is not necessarily limited to a barcode, but may include information such as an OCR in the form of text. The information included in the barcode or OCR of the first barcode area (120) may be, but is not necessarily limited to, at least one of a type, a manufactured date, and an effective date of urine test strip (100). The first barcode area (120) is located at the front of the urine test strip (100) so that the urine test device is the first to see the entire information on the urine test strip (100).

The sector (130) is a part that is discolored by contact with urine, and comprises at least one sector (130, 130′, 130″) connected consecutively. Details of the sectors (130) will be described later.

The first mark (140) may indicate that the number of unused sectors (130) is below a certain quantity. In this case, the certain quantity may be two, but is not necessarily limited thereto. In one embodiment, when the urine test device recognizes the first mark (140) of the urine test strip (100), it may determine that there are two remaining sectors (130) of the urine test strip (100) and request replacement of the urine test cartridge (10). The first mark may be, but is not necessarily limited to, a mark on the separation line (160).

The second mark (150) may indicate that the number of unused sectors (130) is below a certain quantity. In this case, the certain quantity may be, but is not necessarily limited to, one. In one embodiment, when the urine test device recognizes the second mark (150) of the urine test strip (100), it may determine that there is one remaining sector (130) of the urine test strip (100), request replacement of the urine test cartridge (10), and pause operation of the urine test device after using the last sector (130). The second mark may be, but is not necessarily limited to, a mark displayed above the separation line (160). The first mark (140) and the second mark (150) may be displayed on the urine test strip (100) as any hole or print, and may be formed in any form or shape that is readily recognizable by the urine test device.

The separation line (160) may separate the first barcode area (120) from the first sector (130) or the sector (130) from the next sector (130) or the last sector (130) from the spar area (170). The separation lines (160, 160′, 160″, 160″, 160″) may be formed of a material that allows for good separation, so that they may be separated with a small physical force, such as scissors or hands.

The spare area (170) refers to the area behind the last sector (130) that is not filled with anything, and is designed to provide a certain length of free area at the far end of the urine test device (100) so that the last sector (130) may be used. The spare area (170) is necessary to ensure that the urine test device accurately recognizes the test portion of the last sector (130).

The holes (180) are a plurality of holes positioned on at least one end of the urine test strip (100) to be fixed within the urine test device. The holes (180) are provided to allow the urine test strip (100) to move stably within the urine test device. The shape of the holes (180) may be quadrangle, circle, triangle, or any other shape of hole that may be fixed. However, if the urine test device has a structure that moves by holding both sides with rollers made of soft rubber, the holes (180) may be omitted.

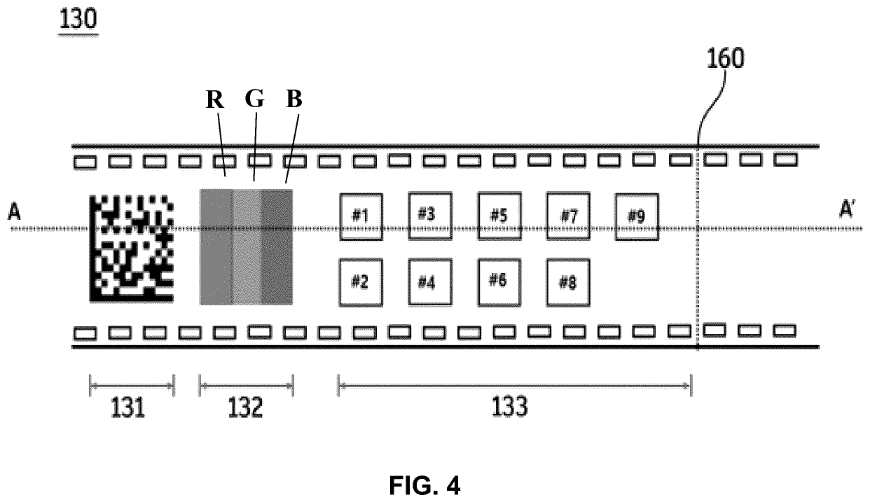

FIG. 4 is a top view of a sector according to an embodiment of the present invention.
Referring to FIG. 4, the sector (130) comprises a second barcode area (131), a standard color display unit (132), and a sample area (133).

The second barcode area (131) may contain a barcode comprising information about the corresponding sector (130), which is not necessarily limited to a barcode but may contain information such as OCR in the form of text. The information contained in the barcode or OCR of the second barcode area (131) may include the same content as the barcode or OCR information of the first barcode area (120), and may further include additional information. The information contained in the barcode or OCR of the second barcode area (131) may include at least one of the following: date of manufacture (or lot #), part #, number of elements (parameters) to be tested per sector, maximum array rows, array columns, cleaning sector identification, and position on the reel (200). The barcode in the second barcode area (131) may be at least one of a one-dimensional code or a two-dimensional code, and if a one-dimensional code is used, it may be a shortened and error-checkable Type C code using only the digits of Code 128. If a two-dimensional code is used, it has the advantage of reducing the area and length compared to a one-dimensional code and compensating for errors. However, the two-dimensional code may be used if the urine test device has a higher information processing capacity. Additionally, at least one of the following may be used as a two-dimensional code: data matrix or QR Code, but not necessarily limited thereto, and any code that is small enough to generate and compensates for errors may be used. When OCR is used for the second barcode area (131), it may be easier for humans to recognize, but may require a urine test device with greater information processing capacity than a two-dimensional code.

The format of the barcode according to one embodiment of the second barcode area (131) may be based on an 8-digit manufactured date YYYYMMDD, an 8-digit effective date or period YYYYMMDD, a 3-digit product name or number, a 2-digit number of elements to be tested per sector (00=sector for cleaning), a 1-digit maximum umber of array rows, a 2-digit number of array columns, and a 1-digit position on the reel (0=start, 1=normal, 2=signal that it is running out, 3=last). This allows interpret US2023081020251231777092041, which is the decoded content of the barcode located in the second barcode area (131), to understand that it was ‘created on Aug. 10, 2023, has an effective date of Dec. 31, 2025, has a product number of 777, contains nine elements, has a maximum of two elements placed in a column, has four columns, and still has plenty of spares on one reel (200).

The standard color display unit (132) is an area for determining a color reference value, which may include the reference color bar RGB (Red, Green, Blue). The standard color display unit (132) is inserted into each sector (130) to compensate for the color reference value, and the urine test device may accurately determine the color change of the sample in the sample area by referring to the color reference value of the standard color display unit (132). The standard color display unit (132) is not necessarily required and may be omitted, as will be described later

The sample area (133) is arranged with samples for testing elements in contact with urine. A total of nine samples are shown in FIG. 4, labeled #1 through #9, but they are not necessarily limited thereto and may be varied depending on how many elements are being tested. Since each element has a different optimal test time (seconds) for urine to come into contact with the sample and discolor, samples testing for elements with shorter optimal test times may be arranged in the front of the test to make efficient use of test time. In this case, the front is the side where the second barcode area 131, which enters the urine test device first, is located.

Below, an embodiment of testing for 10 elements through the sample area (133) will be described.

TABLE 1

| Order of | | |
|---|---|---|
| arrangement | Elements (Parameters) | Optimal testing time |
| 1 | Sugar (Glucose) | 30 seconds |
| 2 | Bilirubin | 30 seconds |
| 3 | Ketone | 40 seconds |
| 4 | Ascorbate | 40 seconds |
| 5 | Specific Gravity | 45 seconds |
| 6 | Blood | 60 seconds |
| 7 | Acidity (pH) | 60 seconds |
| 8 | Protein | 60 seconds |
| 9 | Urobilinogen | 60 seconds |
| 10 | Nitrite | 60 seconds |

Referring to Table 1 above, the arrangement order is made according to the optimal testing time, wherein the optimal testing time may vary depending on the area and thickness of each sample. In addition, the area and thickness of each sample are preferably the same for each element, so that each sample is based on a width of 5 mm and a length of 5 mm, and the height is based on an average of 0.5 mm, but slight variations may occur depending on the element. However, there are no rules specified for the shape of the elements, their width, length, height, or distance between each other, etc., and as long as the measurements are uniform, it is possible to make them freely. Elements with the same optimal testing time may be put in the same group and tested in one column to shorten the length. According to Table 1, ‘sugar’ and ‘bilirubin’ have the same optimal testing time of ‘30 seconds’, so they may be placed in one line.

In FIG. 4, the sample area (133) is shown to have a maximum of two array rows per column, but is not necessarily limited thereto. However, if more than a maximum of three rows are arranged in one column, the height of the reel (200) or the volume of the urine test device should be increased. The distance between each sample placed in the sample area (133) may be, but is not necessarily limited to, at least an average of 2.0 mm to prevent interference with other elemental tests after contact with the urine, and it may be freely fabricated according to measurements. Furthermore, in order to prevent interference with the testing of each element in the sample area (133), a wall may be erected between each sample to physically protect it. Also, if the information in the second barcode area (131) is a cleaning/washing sector, the sample area (133) may not contain a sample, but a substance having a washing action, such as washing alcohol, which may be inserted into the urine test device and used to clean at least one part of the sensors or rollers inside the urine test device. If a substance that acts as a cleaning agent is placed in the sample area (133) to clean the urine test device, it may be used when the sensor of the urine test device is clogged with debris that prevents accurate readings.

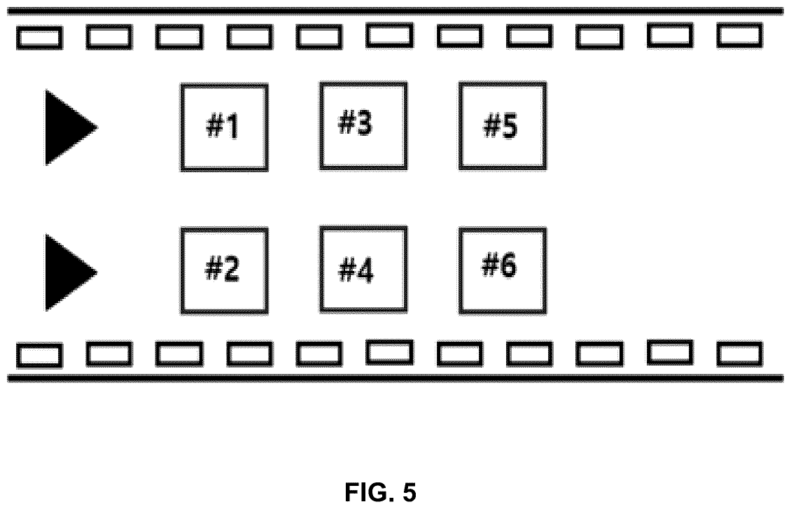

FIG. 5 is a top view of another sector according to another embodiment of the present invention.

Referring to FIG. 5, an illustration of a sector (130) with the second barcode area (131) and the standard color display unit (132) omitted is shown. In case the omission of the second barcode area (131) and the standard color display unit (132) makes it difficult to distinguish between each sector (130), specific marks may be added to indicate the distinction. In this case, in FIG. 5, the specific marks are represented by two triangles. As shown in FIG. 5, the length of one sector (130) may be reduced by omitting the second barcode area (131) and the standard color display unit (132).

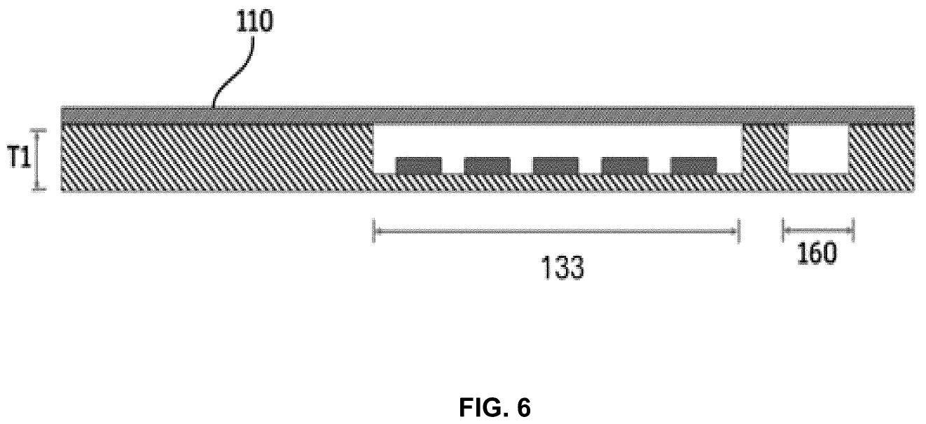

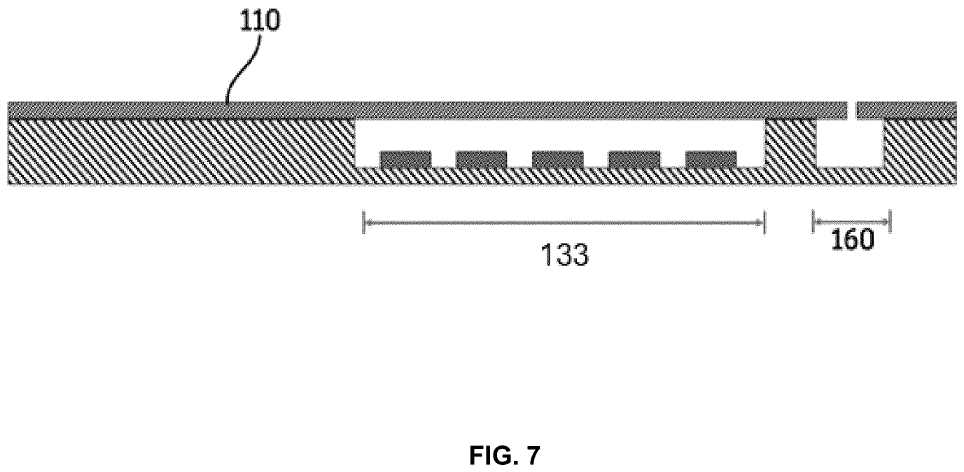

FIG. 6 is a cross-sectional view of a urine test strip according to an embodiment of the present invention, and FIG. 7 is a cross-sectional view of a urine test strip according to another embodiment of the present invention.

Referring to FIG. 6, a side view of the urine test strip (100) with the protective cover (110) attached is shown, where T1 denotes the height of the urine test strip (100). The smaller the height T1 of the urine test strip (100), the more urine test strips (100) may be wound onto the reel (200). The sample area (133) of the urine test strip (100) may be spaced apart from the protective cover (110) at a certain distance to prevent the sample from being contaminated by the protective cover (110). Also, the separation line (160) of the urine test strip (100) may be formed to be thinner than the thickness (T1) of the urine test strip (100) for easy separation from the next sector (130). Referring to FIG. 6 and FIG. 7, the protective cover (110) may be formed with breaks at each position of the separation line (160) in each sector (130) to facilitate smooth separation of the separation line (160) of the urine test strip (100), as shown in FIG. 7.

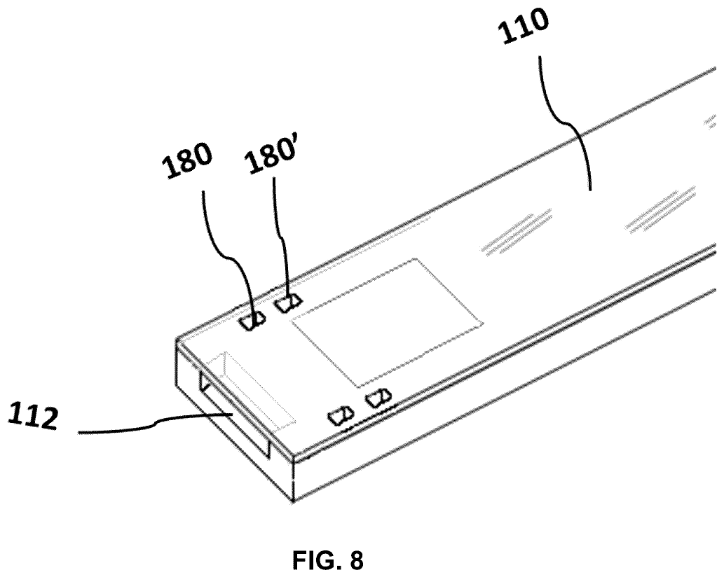

FIG. 8 is a perspective view of a urine test strip according to an embodiment of the present invention.

FIG. 8 is a perspective view of the urine test strip (100), and at least one hole (180, 180′) is formed in the urine test strip (100) and the protective cover (110). However, if the urine test device has a structure that moves by holding both sides of the urine test strip (100) with rollers or the like made of soft rubber, the hole (180) may be omitted. Also, the urine test strip (100) has a starting part (112). The starting part (112) is three-dimensionally formed at the beginning of the urine test strip (100), such that the space is designed to facilitate the removal of the urine test strip (100) and the protective cover (110) when the urine test strip (100) is inserted into the urine test device.

The invention has been described with reference to the embodiments shown in the drawings, but these are exemplary only, and those having ordinary knowledge in the art to which the invention pertains will understand that various modifications and other equivalent embodiments are possible therefrom. Accordingly, the true scope of technical protection of the present invention should be determined by the technical ideas of the attached registration claims.

1. The cartridge for urine test comprising:
a urine test strip being connected to at least one sector that discolors upon contact with urine and having a removable protective cover on a top of the urine test strip; and
a reel on which the urine test strips are wound in a circular shape,
wherein the urine test strip comprises:
a first barcode area where a barcode containing an information about the urine test strip is located;
the at least one sector that discolors when in contact with urine;
a first mark indicating that the number of unused sectors is below a predetermined quantity; and
a second mark indicating that the number of unused sectors is below a predetermined quantity,
wherein the at least one sector comprises
a second barcode area where a barcode containing information about the corresponding sector is located;
a sample area where samples are arranged to test for elements in contact with urine; and
a standard color display unit for determining color reference values.

2. The cartridge of claim 1, wherein at least one fixing hole that is secured within an urine test device is located at least one end of the urine test strip.

3. The cartridge of claim 1, wherein the at least one sector is a plurality of sectors, and a separation line separates each of the plurality of sectors.

4. The cartridge of claim 1, wherein the sample area comprises at least two samples arranged in a column.

5. The cartridge of claim 1, wherein when the sector is for cleaning, the samples of the sample area are replaced with a substance having a washing action.

6. The cartridge of claim 1, wherein walls are positioned between each sample in the sample area to prevent interference between samples.
